(19) United States
(12) Patent Application Publication
(10) Pub. No.: US 2026/0015333 A1
(43) Pub. Date: Jan. 15, 2026

(54) COMPOUNDS AND METHODS FOR INHIBITION OF THE EVOLUTION OF ANTIBIOTIC RESISTANCE

(71) Applicant: VANDERBILT UNIVERSITY, Nashville, TN (US)

(72) Inventor: Houra MERRIKH, Nashville, TN (US)

(21) Appl. No.: 18/992,531

(22) PCT Filed: Jul. 7, 2023
(86) PCT No.: PCT/US2023/069783

§ 371 Date: Jan. 8, 2025

Related U.S. Application Data
(60) Provisional application No. 63/359,379, filed on Jul. 8, 2022.
(60) Provisional application No. 63/377,109, filed on Sep. 26, 2022.

(51) Int. Cl. C07D 307/52; A61K 31/341; A61K 45/06; A61P 31/04

(52) U.S. Cl. CPC C07D 307/52; A61K 31/341; A61K 45/06; A61P 31/04

(57) ABSTRACT

Disclosed is a compound ARM-1 and methods of treating a subject with a bacterial infection or preventing a bacterial infection therewith. ARM-1 functions to prevent the evolution of antimicrobial resistance in bacteria by targeting an evolability factor, Mfd, to prevent the development of antibiotic resistance. By inhibiting the evolution of antibiotic resistance, ARM-1 can be clinically administered to an array of subjects to prevent acceleration and advancement of bacterial infections.

### CROSS REFERENCE TO RELATED APPLICATIONS

This application claims the benefit of priority to U.S. Provisional Application 63/359,379, filed Jul. 8, 2022, and U.S. Provisional Application 63/377,109, filed Sep. 26, 2022, each of which are incorporated by reference herein in their entireties.

### ACKNOWLEDGEMENT OF GOVERNMENT FUNDING

This invention was made with government support under Grant No. R01-AI-127422 awarded by the National Institutes of Health. The government has certain rights in the invention.

### BACKGROUND

Antimicrobial resistance (AMR) has become a major problem in the healthcare field. AMR occurs when bacteria, viruses, fungi, and parasites evolve over time and stop responding to therapeutic drugs, making such infections much more difficult to treat. The World Healthcare Organization (WHO) has declared AMR one of the top 10 global health threats and has projected it to become an extremely costly burden on the world if left unchecked. Drug-resistant bacteria have evolved as “superbugs” that can cause untreatable and highly contagious infections to already vulnerable patients. By prolonging hospital stays, spreading to healthcare personnel, and increasing the risk of routine medical procedures, superbugs can quickly and easily cripple medical centers.

Despite decades of high-throughput screening approaches, in both industrial and academic settings, the results of finding a solution have been limited with no new antibiotics having been identified, and the number of synthetic derivatives that have been developed being relatively limited. Typically, different drugs used to inhibit microbial growth act by inhibiting different targets. However, these therapeutic drugs often target the essential processes in bacteria and fail to address the root of the problem: mutagenesis and subsequent evolution.

To overcome the shortcomings of previous solutions, compounds and methods that target an evolvability factor that is highly conserved across many pathogens is needed. By inhibiting the evolution of a targeted microbe, AMR development in bacteria and other pathogens can be fettered. The compounds and methods disclosed herein address these and other needs.

### SUMMARY

Provided herein is a compound and method for controlling and inhibiting bacterial evolution. The disclosed compound targets a highly conserved evolvability factor. The disclosed compound can prove useful in vivo and in vitro, as the inhibition of bacterial evolution has distinct advantages in the medical and clinical fields.

In one aspect, disclosed herein is the compound, ARM-1, having the formula below (see also FIG. 1B). In some embodiments, pharmaceutically acceptable salts and derivatives of ARM-1 are also disclosed.

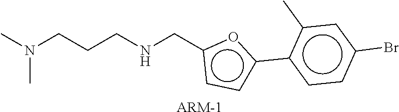

Further, disclosed herein are methods for inhibiting evolution of antimicrobial resistance in bacteria comprising administering to a subject an effective amount of ARM-1 or a salt or derivative thereof. In one example, the bacteria is a drug resistant bacteria. Some examples of drug resistant bacteria that can be targeted are Staphylococcus aureus and Mycobacterium tuberculosis.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF DRAWINGS

The accompanying figures, which are incorporated in and constitute a part of this specification, illustrate several aspects described below.

FIGS. 1A-1C depict an in vivo high throughput screen identifing lead compound ARM-1. FIG. 1A is a schematic of in vivo screen design. In the upper panel, when Mfd is present, RNAP complexes stalled at the lacI-bound operator sequence are quickly removed, resulting in little to no transcription of the lux operon and low luminescence output. In the lower panel, when Mfd is either absent or inhibited, RNAP complexes stalled at the operator are not removed and are able to proceed during temporary dissociation of LacI, resulting in relatively high levels of luminescent output. FIG. 1B depicts the structure of the compound, ARM-1. FIG. 1C depicts translocase activity assay showing relative luminescent output for WT and Δmfd S. typhimurium ST19 strains containing the in vivo system with increasing concentrations of ARM-1. Relative luminescence is normalized to no compound, solvent control, and 0.5% DMSO. *p<0.05 ***p<0.001.

FIGS. 2A-2D depict how ARM-1 affects Mfd's biochemical activity. FIG. 2A depicts how the k_{d} of ARM-1 was determined using microscale thermophoresis. Data shown represent a minimum of three independent experiments. FIG. 2B depicts a NADH-coupled ATPase assay. ATP hydrolysis by Mfd was coupled to NADH oxidation. Phosphoenolpyruvate is converted to pyruvate by pyruvate kinase, transferring a phosphate group onto ADP, which is only present in the reaction following ATP hydrolysis by Mfd. Pyruvate is then converted to lactate by lactate dehydrogenase, oxidizing NADH to NAD⁺. Absorbance at 340 nm by NADH is used to monitor the reaction. Data shown represent a minimum of 3 biological replicates. FIG. 2C depicts a transcription roadblock assay. A P32-labled 176 bp PCR fragment was incubated with E. coli RNAP along with saturating concentrations of ATP, GTP, and UTP. 100 nM S. typhimurium ST19 Mfd preincubated with indicated concentrations of ARM-1 was added and the reaction allowed to proceed for 6 minutes. DNA products were then resolved on a polyacrylamide gel and analyzed by phosphorimaging. Lane 1 shows no enzyme and no compound control. On the left gel, Lanes 2 and 3 show RNAP alone and RNAP with Mfd, respectively, both in the absence of ARM-1 treatment. Lanes 4 shows RNAP and Mfd in the presence 12.5 μM ARM-1. On the right gel, Lane 1 shows the no enzyme and the no compound control, Lane 2 shows RNAP alone, and Lane 3 shows RNAP with 12.5 μM ARM-1. Quantification of these gels is shown below each lane. Analysis was performed using Image Lab 6.0.1. Data shown representative of at least two separate experiments. FIG. 2D depicts a ChIP-qPCR of RpoB enrichment at 16S rDNA. S. typhimurium ST19 of indicated genotype grown to mid exponential phase in cells treated with indicated concentration of ARM-1 for 2 generations prior to harvest. The ChIP were performed using 8RB13 monoclonal antibody against RpoB. Data shown are representative of 3 biological replicates. *p<0.05.

FIGS. 3A-3C depict how ARM-1 reduces mutation frequency in culture and during infection. FIG. 3A depicts a Luria-Delbruck fluctuation assay with ARM-1 treatment. Single colonies of WT and Δmfd S. typhimurium ST19 were used to inoculate overnight cultures with appropriate antibiotic selection. Cultures were then diluted back to OD₆₀₀ 0.0005 and grown to OD₆₀₀ 0.5 with or without 100 μM ARM-1 treatment. Samples were plated on 80 ng/ml ciprofloxacin and grown overnight, then enumerated the following day. Data shown are the result of a minimum of 50 replicates. FIG. 3B depicts the invasion and infection efficiency in the presence of ARM-1. WT S. typhimurium ST19 in mid-log phase growth was used to infect HeLa cells. Invasion was allowed to proceed for 1 hour before uninternalized bacteria were removed and fresh media applied, containing vehicle control or 31.25 μM ARM-1. At the indicated timepoints, HeLa cells were lysed using 1% Triton-H₂O and intracellular bacteria serially diluted and plated on LB for CFU enumeration. FIG. 3C depicts mutation frequency post mammalian cell infection. WT and Δmfd S. typhimurium ST19 were used to infect HeLa cells as described in FIG. 3B. Following infection, intracellular bacteria were harvested and plated on 50 μg/mL 5-fluorocytosine and grown overnight. Mutants were enumerated the following morning. Mutation frequency is shown as mutants per 10⁵ bacteria harvested from HeLa lysates. Data shown are the result of a minimum of 3 biological replicates. **p<0.01

FIGS. 4A-4C depict how ARM-1 inhibits the evolution of antibiotic resistance. FIG. 4A depicts the minimum inhibitory concentration of ARM-1 in E. coli and S. aureus. FIGS. 4B-4C depict the evolution of indicated species against (FIG. 4B) rifampicin and (FIG. 4C) trimethoprim in the presence of ARM-1. Heatmaps show median MIC over time. Concentrations are indicated to the right of each plot. Each strain and antibiotic is the result of at least 12 biological replicates. Concentration of ARM-1 used against L. monocytogenes, P. aeruginosa, and S. aureus is 100 μM. Concentration of ARM-1 used against S. typhimurium is 50 μM. Populations of bacteria are grown overnight in a series of increasing antibiotic concentrations, with or without ARM-1 present. The following day, the population from the highest tolerated concentration is re-challenged with higher concentrations of antibiotic. The median MIC is determined after each 24 hour growth period as the lowest concentration of antibiotic at which at least 50% growth impairment is observed compared to an untreated population. MIC fold change is reported as the change in MIC from the first day of growth to each subsequent day.

FIGS. 5A-5D depict additional information regarding ARM-1. FIG. 5A depicts the workflow of Calibr library screening. FIG. 5B depicts a luminescence response curve from ARM-1 performance in original screen in the presence of Mfd and active transcription. FIG. 5C depicts a luminescence response curve from ARM-1 performance in original screen in the absence of Mfd. FIG. 5D depicts antibacterial activity of ARM-1 in original screen.

FIG. 6 depicts NADH-Coupled ATPase assay control conditions. 100 μM ARM-1 was incubated with repair assay buffer supplemented with 4.4 units pyruvate kinase, 5.7 units lactate dehydrogenase, 500 μM phosphoenolpyruvate, and 50 nM Mfd, in the presence or absence of NADH and ATP.

FIG. 7 depcits ChIP-qPCR of rpoB enrichment at 23S rDNA. S. typhimurium ST19 of indicated genotype grown to mid exponential phase and treated with indicated concentration of ARM-1 for 2 generations prior to harvest. IP performed using 8RB13 monoclonal antibody against rpoB. Data shown representative of 3 biological replicates.

FIG. 8 depicts a proposed model of ARM-1 effect on interaction between Mfd and stalled transcription elongation complexes. Left panel shows proposed behavior of Mfd and RNAP in the absence of ARM-1. Increased population of initiation complexes (ICs) and decreased elongation complexes (ECs) after the addition of Mfd (FIG. 2C, left panel, lane 3) suggests that after Mfd displaces RNAP from the DNA template, RNAP is “recycled” and returns to the promoter to re-initiate transcription. Right panel shows proposed behavior of Mfd and RNAP in the presence of ARM-1. Under these conditions, a decrease in EC population suggests that Mfd does displace stalled RNAPs, but lack of a simultaneous increase in IC population suggests that RNAP is not effectively recycled to the promoter and does not reinitiate transcription under these conditions.

FIGS. 9A-9B depict the cytotoxicity of ARM-1 against indicated mammalian cell lines. FIG. 9A depicts toxicity reported from original Calibr screen data against HEK293T and HepG2 cell lines. FIG. 9B depicts toxicity determined using Promega CellTox reagents following 8 hours of exposure of HeLa, Caco-2, and HEK293 cells to varying concentrations of ARM-1. Relative fluorescent units (RFUs) reported relative to solvent and no substrate controls.

FIG. 10 depicts growth curves of indicated species in the presence of increasing concentrations of ARM-1. Precultures of each species were grown with appropriate antibiotic selection, then diluted back to OD600 0.05 in a 96 well plate. Indicated concentrations of ARM-1 were added, and plates incubated overnight at 37° C. with shaking. OD measurements taken every 60 s by BioTek plate reader. Data shown are representative of at least 6 biological replicates. Growth curve for S. typhimurium also confirms MIC of 400 μM ARM-1 for this species, with no growth observed over the experimental timeframe.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint.

It is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined.

The term “patient” refers to a human in need of treatment for any purpose. However, the term “patient” can also refer to non-human animals, preferably mammals such as dogs, cats, horses, cows, pigs, sheep, and non-human primates, among others, that are in need of treatment.

By “reduce” or other forms of the word, such as “reducing” or “reduction,” is meant lowering of an event or characteristic. It is understood that this is typically in relation to some standard or expected value, in other words it is relative, but that it is not always necessary for the standard or relative value to be referred to. For example, “reduces bacterial growth” means reducing the rate of growth of bacteria relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

### Compounds

### Compound ARM-1

Disclosed, in one aspect, is the compound N¹-((5-(4-bromo-2-methylphenyl)furan-2-yl)methyl)-N³,N³-dimethylpropane-1,3-diamine, also referred to as “ARM-1.” ARM-1 has the following formula:

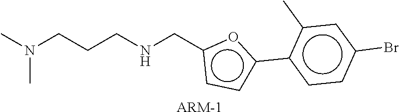

High throughput screens identified small molecules that can permeate bacterial cells and have little or no bactericidal activity or toxicity to mammalian cells. The screen was conducted to reveal Mfd-specific inhibitors, where Mfd is an RNApolymerase (RNAP)-associated DNA translocase that increases mutagensis and accelerates evolution of AMR. Mfd is a mutation frequency decline protein. The identified ARM-1 is thus a compound that can be administered as an Mfd-specific inhibitor. ARM-1 binds to Mfd with a k_{d} of 4.25 μM (FIG. 2A), which allows administration of the compound in relatively low concentrations. References to ARM-1 throughout this disclosure mean ARM-1 or a pharmaceutically acceptable salt or a derivative thereof, unless stated to the contrary.

Pharmaceutically acceptable salts include compounds wherein the parent compound ARM-1 is modified by making an acid or base salt thereof, and further refers to pharmaceutically acceptable solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from inorganic or organic acids. For example, conventional acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)n—COOH where n is 0-4, and the like. The pharmaceutically acceptable salts of can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable.

### Methods

### Development of ARM-1

Antimicrobial resistance (AMR) is among the top five global health crises of the modern world. World Health Organization priority pathogens such as methicillin-resistant Staphylococcus aureus (MRSA) and drug-resistant Mycobacterium tuberculosis (DR-TB), along with hundreds of other drug resistant bacterial species, account for global mortality rates over 10 million every year (Dadgostar 2019). By 2050, if unchecked, AMR is projected to cost 210 trillion USD in annual global GDP, with roughly 16.7 trillion from DR-TB alone.

Previously, a shift in the approach to combatting AMR was proposed. It was proposed that inhibiting evolution by directly inactivating the mechanisms that increase mutation rates could prevent the development of AMR during the treatment of infections. Previous work showed that Mfd, an RNA polymerase (RNAP)-associated DNA translocase, increases mutagenesis and accelerates evolution of AMR across highly divergent species. In this work, the discovery of a small molecule that inactivates the evolvability factor, Mfd, is described, demonstrating that targeting bacterial evolvability factors directly is possible and can indeed prevent the development of AMR during treatment of infections.

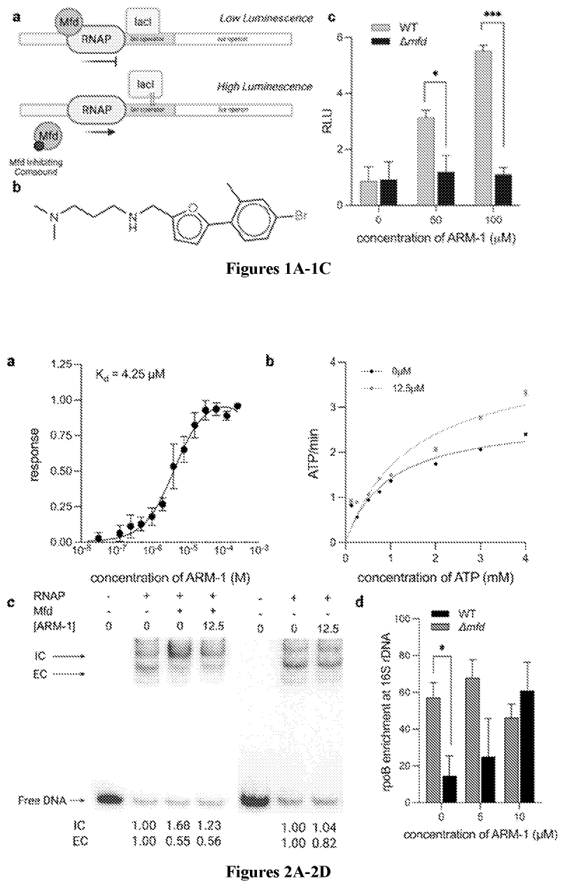

In order to determine if Mfd's activity and/or RNAP termination function could be inactivated through the use of a small molecule, a high-throughput in vivo screen of roughly 250,000 compounds was conducted. To identify such compounds, a previously described system to facilitate the screen that would reveal Mfd-specific inhibitors was modified (FIG. 1A and FIGS. 5A-5D). The screen was designed such that it would only identify small molecules that are able to permeate bacterial cells and have little or no bactericidal activity or toxicity to mammalian cells. In this screen, Δmfd Escherichia coli cells containing a reporter system encoded on two different plasmids was used. The reporter system consists of one plasmid with a lac operator (lacO) encoded directly upstream of the lux operon which expresses proteins that produce luciferase, leading to easily detectable luminescence. The second plasmid expresses the Salmonella typhimurium Mfd protein using the inducer IPTG. The LacI protein which binds to lacO is a barrier to transcription; RNAP is unable to readily pass the bound protein and therefore, the transcription machinery stalls. The stalled RNAP is quickly recognized by Mfd and is pushed off the DNA. However, based on simple biophysical properties, LacI “breathes” on and off DNA at a measured rate. If Mfd is not present, or its activity is inhibited by for instance a small molecule, the stalled RNAP will be able to proceed through the lux operon during the temporary dissociation of LacI from the operator sequence lacO, leading to the production of luminescence (FIG. 1A). Using this property, relative luminescence in the presence of each compound to look for Mfd inhibition in this in vivo screen was measured (FIG. 1A and FIGS. 5A-5D). To ensure that the observed changes were not due to potential off target effects, in parallel, strains containing plasmids that did not contain the mfd gene were used and also examined IPTG-dependence of the observed changes. Positive hits from this system are those which result in high luminescence only when transcription is active and Mfd is present, and do not show prohibitive bactericidal or cytotoxic effects (FIGS. 5A-5D and FIGS. 9A-9B). This approach led to the discovery a compound that inhibited Mfd, which is hereby referred to as ARM-1 (Anti-Resistance Molecule 1). The structure of ARM-1 (VU0943184) is shown in FIG. 1B.

As many commercially available compounds can have impurities, ARM-1 was synthesized to above 99% purity and repeated the experiments performed in the screen prior to characterization of the compound's downstream effects on Mfd function. For the experiments, this compound was synthesized following an original synthesis strategy, and the product was thoroughly validated prior to proceeding (for synthesis description and compound validation, see Supplemental Information). It was found that ARM-1 binds to Mfd with a k_{d} of 4.25 μM (FIG. 2A), a notably tight binding affinity for a lead compound prior to medicinal chemistry optimization. This allowed the use of relatively low concentrations of ARM-1 in in vivo assays, effectively circumventing any potential bactericidal or cytotoxic effects. Using the same system employed for the high throughput screen, the effect of ARM-1 on Mfd's translocase activity was measured and confirmed. Treating cells with 50 to 100 μM ARM-1 resulted in a 3- to 5-fold inhibition of translocase activity in vivo. Given that there was no effect of ARM-1 on luminescence in cells lacking Mfd, the effects of ARM-1 are concluded to indeed directly through inhibition of Mfd (FIG. 1C).

Mfd translocates along double-stranded DNA and dislodges stalled RNAP complexes. Since both processes have been demonstrated to be dependent on Mfd's ability to hydrolyze ATP, the effect of ARM-1 on in vitro ATP hydrolysis by Mfd directly was examined. At dosages ranging from 6.25 μM to 50 μM ARM-1, at ratios of 125 to 1000 ARM-1 per Mfd, a dose dependent increase of ATP hydrolysis was observed (FIG. 2B). In the presence of ARM-1, ATP turnover by Mfd ilwas not inhibited. If anything, the ATPase activity was enhanced almost 3-fold in the presence of 50 μM ARM-1 (FIG. 2B and Table 1). Together, these results suggest that Mfd acts on RNAP not through inhibition of enzymatic activity but also through an allosteric conformational change, which is common to transcription terminators such as Rho (REF). To further investigate the mechanism by which ARM-1 inhibits Mfd activity, a slightly modified version a of previously described in vitro RNA polymerase displacement assay was performed. In this assay, E. coli RNAP holoenzyme is stalled by CTP starvation on a radiolabeled, short DNA fragment containing a constitutively active promoter. The first cytosine is encoded on this DNA fragment 21 nucleotides downstream of the promoter. In the absence of CTP, RNAP stalls at this location. This stalled RNAP can then be removed by Mfd. As has been observed previously, when this displacement assay was performed with purified proteins, two DNA-protein complexes were observed by electrophoretic mobility shift assays (EMSAs); a slower migrating initiation complex (IC), and a faster migrating elongation complex (EC). When quantified, a ratio of EC to IC of approximately 1 was observed, which was consistent with prior work. Addition of purified Mfd removed the stalled RNA polymerase, which results in a reduction the ratio of ECs compared to ICs. It is reasoned that when removed by Mfd, the displaced RNAP is free to re-bind the promoter, which is apparent by the increase in the amount of ICs (FIG. 2C, EC/IC=0.33). In contrast, when Mfd is pre-incubated with ARM-1, though a decrease in the amount of EC when compared to the reaction without Mfd is still observed, the corresponding increase in ICs (EC/IC=0.46) is no longer observed, which is normally a downstream result of Mfd's RNAP removal function. This indicates that ARM-1 indeed inhibits Mfd's function by preventing its ability to remove stalled RNAPs from DNA. The reduction in the ECs in the presence of ARM-1 is most likely due to the fact that at equilibrium, when RNAP cannot dissociate from DNA, it cannot re-initiate transcription, consequently preventing new EC formation. The effects of ARM-1 in these experiments are not due to off target effects on RNAP. In control reactions, ARM-1 did not have any detectable changes on RNAP complexes in the absence of Mfd (FIG. 2C).

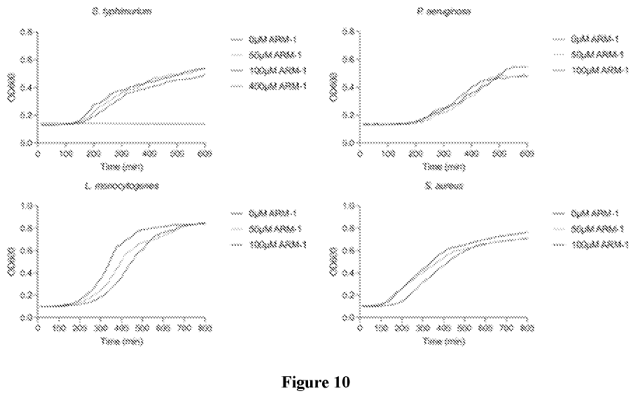

As a follow-up to the in vitro experiments, the effect of ARM-1 on Mfd in vivo was investigated. For this, chromatin immunoprecipitations of the β subunit of RNAP (RpoB) in WT and Δmfd S. typhimurium ST19 strains treated with ARM-1 for at least two generations of exponential growth is performed. In the untreated condition, an increase in RpoB association with rDNA, a highly transcribed region of the bacterial genome, in the absence of Mfd is observed (FIG. 2D and FIG. 7). This effect is consistent with Mfd's function in displacing stalled elongation complexes, particularly at regions that are difficult to transcribe. At 5 mM ARM-1, there is little change in RpoB association, but at 10 mM there is a dramatic decrease in association of RpoB in WT cells, consistent with Mfd inhibition and extended occupancy of elongation complexes at the target region (FIG. 2D and FIG. 7). In the Δmfd background, RpoB enrichment is not different from WT in the absence of ARM-1. This suggests, consistent with observations from the transcription roadblock assay in vitro, that ARM-1 may have a limited effect on elongating RNAP complexes independent of Mfd (FIG. 2C right panel and FIG. 2D). Because no growth defects at these concentrations of ARM-1 is observed, these patterns are unlikely attributable to indirect effects (FIG. 10). In summary, these data support a nuanced effect of ARM-1 on Mfd's biochemical activity both in vitro and in vivo.

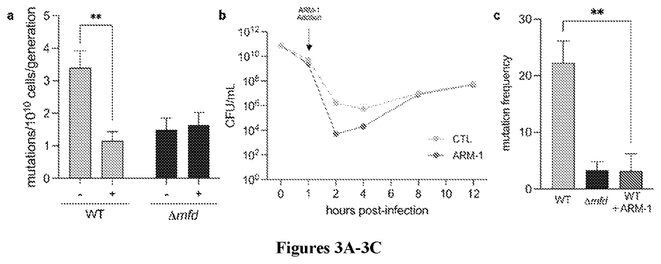

Previously it has been shown that the mutagenic effect of Mfd is conserved during bacterial infection of eukaryotic cells. Luria-Delbruck assays have demonstrated that mutation rates are increased by Mfd. With ARM-1 treatment, a 3-fold reduction in WT mutation frequency is observe, consistent with direct inhibition of Mfd (FIG. 3A). To determine if ARM-1 can also reduce mutagenesis during infection, HeLa cells were infected with wild-type and Δmfd S. typhimurium ST19, and measured mutation frequency through acquisition of 5-fluorocytosine resistance. The compound was added to the culture media following invasion to ensure uniform treatment conditions. an almost 7-fold decrease in mutation frequency in the absence of Mfd is observed (FIG. 3C). WT S. typhimurium cells have a mutation frequency of 22.3 mutations per 10⁵ bacteria during infection, whereas Δmfd strains have a 3.3 mutations per 10⁵ bacteria during infection (FIG. 3C). It was found that treatment with ARM-1 reduces WT mutation frequency to 3.2 mutations per 10⁵ bacteria, a 7-fold reduction compared to untreated cells, and almost exactly at the level of Δmfd strains (FIG. 3C). Neither the Δmfd mutant nor ARM-1 or solvent-treated wild-type or Δmfd strains show a defect in invasion or proliferation in HeLa cells compared to untreated WT (FIG. 3B). These observations confirm that the differences in mutation frequencies are not due to altered growth dynamics. Furthermore, no cytotoxic effects of ARM-1 on HeLa cells were observed during the timeframe of this experiment, allowing for direct comparisons between ARM-1 untreated and treated infections (FIGS. 9A-9B). Taken together, these data suggest that ARM-1 is directly inhibiting bacterial mutagenesis mechanisms.

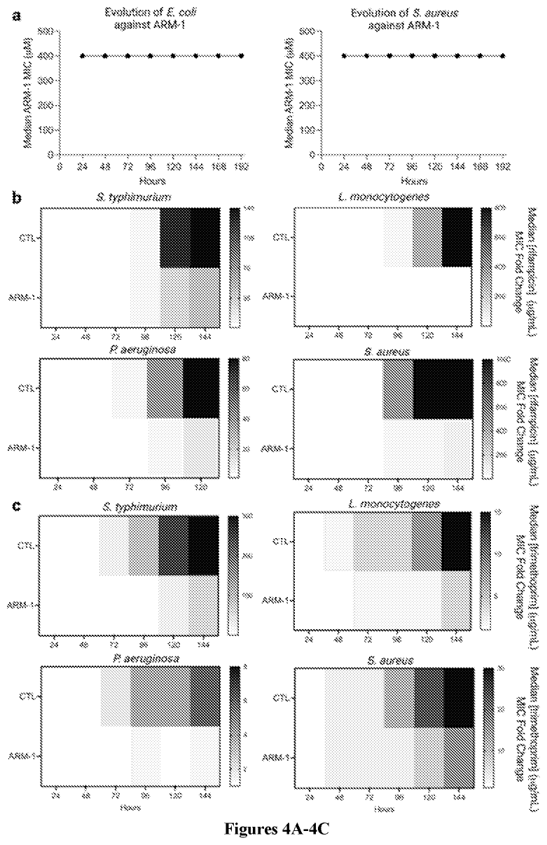

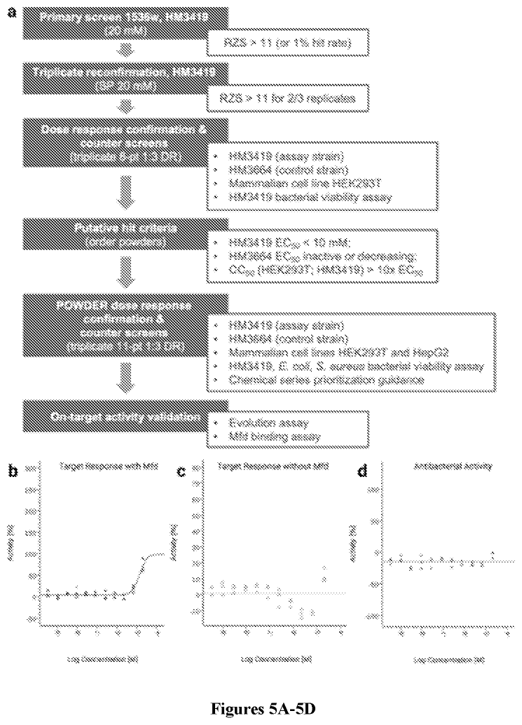

Next, the effect of ARM-1 on the evolution of antibiotic resistance was examined. Genetic deletion of Mfd is known to reduce the rates and extent of resistance development in laboratory evolution experiments. Using an assay previously developed, the impact of ARM-1 was assessed on resistance development across diverse pathogens and in response to multiple classes of antibiotics. In these experiments, bacteria are challenged with increasing concentrations of antibiotic over a minimum of least 55 generations. Reduced resistance development in bacteria treated with ARM-1 was observed compared to those that were untreated, in all species and antibiotics tested. In S. typhimurium, challenged with rifampicin or trimethoprim, or phosphomycin, it was observed at minimum a 100-fold difference in median minimum inhibitory concentration (MIC) fold change between untreated and treated conditions (FIG. 4B). This pattern was also observed in P. aeruginosa, S. aureus, and L. monocytogenes, with ARM-1 treatment resulting in 80, 800, 1000, and P FOLD reduction in resistance to rifampicin, respectively (FIG. 4B). These results were the same when these species were challenged with trimethoprim (FIG. 4C). Sequencing of relevant resistance loci over the course of treatment confirmed that these observations are due to genetic changes; many more resistance mutations arise in untreated populations compared to those treated with ARM-1 (Table 1). Doubling time was consistent between treated and untreated population across all species tested (FIG. 10), indicating that the results are not simply attributable to growth defects. Furthermore, patient isolates of S. typhimurium, S. aureus, and P. aeruginosa which already carry resistance mutations to other antibiotics were used, and still observed a reduction in resistance development during experiments. These results clearly demonstrate that ARM-1 inhibits resistance development in diverse bacterial species. There were no observed changes in MIC or any growth defects when E. coli or S. aureus were challenged with ARM-1 alone; the MIC remained constant at 400 μM for 196 hours of exposure (FIG. 4A). This suggests that bacteria cannot evolve resistance to ARM-1 itself.

In addition to the discovery of ARM-1 and its impact on AMR, it is also noted that this work has provided information regarding how the highly conserved protein Mfd functions. The in vitro (and in vivo) findings show that Mfd not only requires its ATPase hydrolysis activity to remove stalled RNAPs from DNA, but that it also harbors an allosteric mechanism for Mfd to remove RNAPs from DNA. In other words, though not discussed in depth within the literature, it appears that Mfd's allosteric modulation of RNAP is a part of its function. Given that the majority of known transcription terminators use allosteric mechanisms, the new finding on Mfd's mechanistic function is in essence not necessarily surprising. Nevertheless, this finding makes a valuable contribution to the field, and ARM-1 can also be used to study Mfd's mechanistic functions further.

In summary, this work shows that the rise of AMR can be inhibited with a simple anti-evolution drug. There are no new or old therapies that are immune to mutagenesis; the driver of adaptative evolution. When processes are inhibited either through antibiotics or other therapies that kill cells, a genetic screen that selects for mutants that are resistant to the pressure placed on bacterial cells that are causing the infection is performed. Therefore, if mutation rates are not reduced and evolution is not inhibited, the problem of AMR is only going to be exacerbated. Even more problematic is that researchers are discovering new antibiotics and other therapies that kill cells which are providing extremely valuable resources, and yet, these treatments are put under the threat of adaptive evolution by pathogens. The ultimate solution to protecting the new therapeutics and the development of AMR infections is to prevent the ability of bacteria to evolve resistance during treatment. This work shows that this is possible. The discovery of ARM-1 sets the foundation for the development of clinically usable anti-evolution drugs. Such a drug can apparently be used against AMR development in many pathogens which simplifies the process: one anti-evolution drug could be sufficient for usage of many different types of infections by different pathogens and regardless of the antibiotics that are being used.

### Method of Inhibiting the Evolution of Antimicrobial Resistance

In another aspect, disclosed are methods of inhibiting the evolution of antimicrobial resistance (AMR), including administering a therapeutically effective amount of ARM-1, or a pharmaceutically acceptable salt or a derivative thereof, to a subject. In some examples, ARM-1 functions on drug resistant bacteria. In specific examples, the bacteria is Staphylococcus aureus or Mycobacterium tuberculosis. The infection typically comprises a bacterial infection. The infection can be a bacterial that is pathogenic to the subject, or which infects the subject and is pathogenic to a downstream consumer of food products made from the subject. ARM-1 targets the RNA polymerase (RNAP)-associated translocase evovability factor. In specific examples, the RNAP translocase is Mfd.

In some embodiments, the bacterial infection can be caused by, for example, Acinetobacter baumannii, Actinobacillus actinomycetemcomitans, Agrobacterium tumefaciens, Aggregatibacter actinomycetemcomitans, Bacillus (e.g., cereus, anthracis), Bacteroides forsythus, Branhamella catarrhalis, Bordetella pertussis, Borrelia (e.g., burgdorferi, garinii, afzelii, recurrentis), Brucella (e.g., abortus, canis, melitensis, suis), Campylobacter (e.g., jejuni, coli), Candidatus liberibacter, Citrobacter diversus, Chlamydia (e.g., pneumoniae, trachomatis, psittaci), Clostridium (e.g., botulinum, difficile, perfringens, tetani), Corynebacterium diphtheriae, Enterobacter aerogenes, Enterococcus (e.g., faecium, faecalis), Edwardsiella tarda, Escherichia coli, Francisella tularensis, Fusobacterium nucleatum, Haemophilus influenzae, Helicobacter pylori, Klebsiella pneumoniae, Legionella pheumophila, Listeria monocytogenes, Mycobacterium (e.g., tuberculosis, leprae, ulcerans), Mycoplasma pneumoniae, Morganella morganii, Neisseria (e.g., meningitidis, gonorrhoeae), Propionibacterium, Proteus mirabilis, Porphyromonas gingivalis, Pseudomonas aeruginosa, Rickettsia, Salmonella species (e.g., Salmonella enterica), Serratia marcescens, Shigella (e.g., sonnei, boydii), Staphylococcus (e.g., aureus, epidermidis, saprophyticus), Streptococcus (agalactiae, pneumoniae, pyogenes), Treponema pallidum, Ureaplasma urealyticum, Veillonella parvula, Vibrio cholera, Yersinia (e.g., pestis, enterocolitica, pseudotuberculosis), or combinations thereof.

The subject can be any human or animal subject. For example, the subject can be a mammal (e.g., a human, dog, cow, horse, mouse, rabbit, non-human primate, etc.). In some embodiments, the subject is a livestock or farm mammal (e.g., sheep, goat, cow, pig, etc.) or, alternatively, a human. The subject can be a medical patient. The subject can also be a non-mammal animal such as an avian subject (a bird). In some embodiments, the avian subject can be livestock or farm bird, for example poultry. For example, the subject can be a chicken, turkey, quail, duck, emu, goose, ostrich, pigeon, pheasant, rhea, guineafowl, or the like. Other non-mammal animal subjects include insects such as a moth, fly (e.g., fruit fly), beetle, ant, spider, butterfly, mosquito, flea, mantis, termite, cricket, grasshopper, bee, caterpillar, centipede, etc. In some embodiments, the subject is at risk for a bacterial infection, for example by housing in quarters in close proximity to other animals which can be or are infected with a bacterial infection. The subject can be a male or female of any age, size, or other general classifiers.

In some embodiments, ARM-1 can be administered in an in vitro setting. In some examples, ARM-1 has above an 80% purity (e.g., above an 85% purity, above a 90% purity, above a 95% purity, above a 97% purity, above a 98% purity, or above a 99% purity). In some embodiment, ARM-1 can be administered with a concentration of 50 to 100 μM (e.g., a concentration of 50 to 60 μM, a concentration of 60 to 70 μM, a concentration of 80 to 90 μM, or a concentration of 90 to 100 μM).

In some examples, the therapeutically effective amount of ARM-1 can be administered to the subject orally. In further examples, the therapeutically effective amount of ARM-1 can be in a tablet, troche, pill, or capsule. In some examples, the therapeutically effective amount of ARM-1 can be administered buccally. In certain examples, the therapeutically effective amount of ARM-1 can be administered to the subject intravenously. In specific examples, the therapeutically effective amount of ARM-1 can be administered to the subject as a spray. In further examples, the spray is administered nasally. In some examples, the therapeutically effective amount of ARM-1 can be administered to the subject topically. In further examples, the therapeutically effective amount of ARM-1 can be in an ointment, cream, lotion, solution, or tincture.

ARM-1 can be administered either sequentially or simultaneously in separate or combined pharmaceutical formulations. When ARM-1 is used in combination with a second therapeutic agent, the dose of each compound can be either the same as or differ from that when the compound is used alone. Appropriate doses will be readily appreciated by those skilled in the art.

The term “administration” and variants thereof (e.g., “administering” a compound) in reference to a compound disclosed herein means introducing the compound into the system of the subject in need of treatment. When a compound disclosed herein is provided in combination with one or more other active agents (e.g., a cytotoxic agent, etc.), “administration” and its variants are each understood to include concurrent and sequential introduction of the compound or prodrug thereof and other agents.

Administration can be accomplished by any suitable method and technique presently or prospectively known to those skilled in the art. For example, the disclosed compounds can be formulated in a physiologically-or pharmaceutically-acceptable form and administered by any suitable route known in the art including, for example, oral and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

ARM-1, as disclosed herein, and compositions comprising it, can also be administered utilizing liposome technology, slow release capsules, implantable pumps, and biodegradable containers. These delivery methods can, advantageously, provide a uniform dosage over an extended period of time. ARM-1 can also be administered in its crystalline form.

ARM-1, as disclosed herein, can be formulated according to known methods for preparing pharmaceutically acceptable compositions. Formulations are described in detail in a number of sources which are well known and readily available to those skilled in the art. For example, Remington: The Science and Practice of Pharmacy (1995) describes formulations that can be used in connection with the disclosed methods. In general, the compound disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise from 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use.

Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

The compound and compositions disclosed herein can be administered intravenously, intramuscularly, or intraperitoneally by infusion or injection. Solutions of the active agent can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

The compound and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the subject's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

For topical administration, the compound and agents disclosed herein can be applied as a liquid or solid. It will generally be desirable to administer them topically to the skin as compositions, in combination with a dermatologically acceptable carrier, which can be a solid or a liquid. The compound and agents disclosed herein can be applied directly to the growth or infection site.

Useful solid carriers include finely divided solids such as talc, clay, microcrystalline cellulose, silica, alumina, and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508.

Useful dosages of the compound and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex, and extent of the disease in the subject and can be determined by one of skill in the art. The dosage can be adjusted by the individual physician in the event of any counterindications. Dosage can vary, and can be administered in one or more dose administrations daily, for one or several days.

The dose administered to a subject, particularly a human, should be sufficient to achieve a therapeutic response in the subject over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

In some examples, the therapeutically effective amount of ARM-1 can include a pharmaceutical formulation including a combination of ARM-1 and a pharmaceutically acceptable carrier. In specific examples, the pharmaceutically acceptable carrier can include a binder, excipient, disintegrating agent, sweetening agent, lubricant, flavoring agent, inert diluent, assimilable edible carrier, or any combination thereof.

As used herein, the term “pharmaceutically acceptable carrier” refers to sterile aqueous or nonaqueous solutions, dispersions, suspensions, or emulsions, as well as sterile powders for reconstitution into sterile injectable solutions or dispersions just prior to use. Pharmaceutically acceptable carriers can include, but are not limited to, inert diluents, assimilable edible carriers, binders, excipients, disintegrating agents, sweetening agents, lubricants, or flavoring agents. Examples of suitable aqueous and nonaqueous carriers, diluents, inert diluents, solvents, assimilable edible carriers, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents, and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

In some examples, binder can include gum tragacanth, acacia, corn starch, gelatin, or any combination thereof. In further examples, excipients can include dicalcium phosphate. In certain examples, disintegrating agent can include corn starch, potato starch, alginic acid, or any combination thereof. In specific examples, sweetening agent can include sucrose, fructose, lactose, aspartame, or any combination thereof. In some examples, lubricant can include magnesium stearate. In further examples, flavoring agent can include peppermint, oil of wintergreen, cherry flavoring, or any combination thereof. In certain examples, inert diluent can include anhydrous lactose, lactose monohydrate, sugar alcohols, such as sorbitol, xylitol, or mannitol, or any combination thereof. In specific examples, assimilable edible carrier can include polysaccharides, polymers, pectin, polypeptides, or any combination thereof.

In some examples, the therapeutically effective amount of ARM-1 can be from 1 to 5,000 mg per day. Further, the therapeutically effective amount of ARM-1 can be from 1 to 1,000, 1,000 to 2,000, 2,000 to 3,000, 3,000 to 4,000, or 4,000 to 5,000 mg per day. In certain examples, the therapeutically effective amount of ARM-1 can be from 1 to 500, 500 to 1,000, 1,000 to 1,500, 1,500 to 2,000, 2,000 to 2,500, 2,500 to 3,000, 3,000 to 3,500, 3,500 to 4,000, 4,000 to 4,500, or 4,500 to 5,000 mg per day. Further, the therapeutically effective amount of ARM-1 can be from 1 to 200, 200 to 400, 400 to 600, 600 to 800, 800 to 1,000, 1,000 to 1,200, 1,200 to 1,400, 1,400 to 1,600, 1,600 to 1,800, 1,800 to 2,000, 2,000 to 2,200, 2,200 to 2,400, 2,400 to 2,600, 2,600 to 2,800, 2,800 to 3,000, 3,000 to 3,200, 3,200 to 3,400, 3,400 to 3,600, 3,600 to 3,800, 3,800 to 4,000, 4,000 to 4,200, 4,200 to 4,400, 4,400 to 4,600, 4,600 to 4,800, or 4,800 to 5,000 mg per day.

In some examples, the therapeutically effective amount of ARM-1 can be from 1 to 1,000 mg/kg. Further, the therapeutically effective amount of ARM-1 can be from 1 to 200, 200 to 400, 400 to 600, 600 to 800, or 800 to 1,000 mg/kg. In certain examples, the therapeutically effective amount of ARM-1 can be from 1 to 100, 100 to 200, 200 to 300, 300 to 400, 400 to 500, 500 to 600, 600 to 700, 700 to 800, 800 to 900, or 900 to 1,000 mg/kg. Further, the therapeutically effective amount of ARM-1 can be from 1 to 25, 25 to 75, 75 to 125, 125 to 175, 175 to 225, 225 to 275, 275 to 325, 325 to 375, 375 to 425, 425 to 475, 475 to 525, 525 to 575, 575 to 625, 625 to 675, 675 to 725, 725 to 775, 775 to 825, 825 to 875, 875 to 925, 925 to 975, or 975 to 1,000 mg/kg.

In some examples, the therapeutically effective amount of ARM-1 can be from 1 to 200 mg/kg per day. Further, the therapeutically effective amount of ARM-1 can be from 1 to 50, 50 to 100, 100 to 150, or 150 to 200 mg/kg per day. In certain examples, the therapeutically effective amount of ARM-1 can be from 1 to 25, 25 to 50, 50 to 75, 75 to 100, 100 to 125, 125 to 150, 150 to 175, or 175 to 200 mg/kg per day. Further, the therapeutically effective amount of ARM-1 can be from 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, or 190 to 200 mg/kg per day.

### Composition

### Pharmaceutical Composition

Provided herein are pharmaceutical compositions, including ARM-1 and an antibiotic drug. For examples, disclosed are methods of reducing or preventing antimicrobial resistance of bacteria that comprise administering to a patient in need thereof an amount of ARM-1 effective to modulate the antimicrobial resistance of the bacteria. Appropriate doses will be readily appreciated by those skilled in the art. The amount of ARM-1, or a pharmaceutically acceptable salt or a derivative thereof, in the composition can be, in some examples, is therapeutically effective amount. In some examples, ARM-1 can be administered in the same composition as the antibiotic drug. In other examples, ARM-1 can be administered in a separate composition as the antibiotic drug.

In some examples, the antibiotic drug can include penicillins, tetracyclines, cephalosporins, quinolones, lincomycins, macrolides, sulfonamides, glycopeptides, aminoglycosides, carbapenems, or any combination thereof. In some examples, the antibiotic drug can include penicillins. In further examples, the antibiotic drug can include tetracyclines. In certain examples, the antibiotic drug can include cephalosporins. In other examples, the antibiotic drug can include quinolones. In specific examples, the antibiotic drug can include lincomycins. In further examples, the antibiotic drug can include macrolides. In further examples, the antibiotic drug can include sulfonamides. In other examples, the antibiotic drug can include glycopeptides. In certain examples, the antibiotic drug can include aminoglycosides. In specific examples, the antibiotic drug can include carbapenems.

Penicillins, also known as “beta-lactam” antibiotics, consists of aminopenicillins, antipseudomonal penicillins, beta-lactamase inhibitors, natural penicillins, and the penicillinase resistant penicillins. Tetracyclines refer to the broad-spectrum antibiotics that treat many conditions such as acne, urinary tract infections (UTIs), intestinal tract infections, eye infections, sexually transmistted diseases, periodontitis, and other bacterial infections. Cephalosporins refer to the five generations of antibiotics that, as a class, include gram-negative infections and have updated structures. Quinolones, also known as fluoroquinolones, are a synthetic, bactericidial antibacterial class for use in adults when other options have been exhausted. Lincomycins has antibiotic activity against gram-positive aerobes and anaerobes, as well as some gram-negative anaerobes. Macrolides, including ketolides, are a class of antibiotics used to treat community-acquired diseases. Sulfonamides are a class of antibiotics that are effective against many gram-negative bacteria and some gram-positive bacteria, though resistance is widespread. Glycopeptide antibiotics are often used for treating, among other infections, staphylococcus aureus (MRSA) infections. Aminoglycosides are a class of antibacterial drugs that inhibit synthesis by binding to the 30S ribosome and are typically administered intravenously. Carbapenems are also beta-lactam antibiotics that are injectable and are usually saves for more serious infections.

### Nasal Spray

Also provided herein, is a nasal spray including ARM-1. Nasal spray as used herein is a spray composition that is suitable for spraying into one or both nostrils and is safe for contact with mucous membranes within the nasal cavities.

The nasal spray can further include a pharmaceutically acceptable buffer in order to maintain the desired pH. Non-limiting examples of suitable buffers used to adjust and maintain the pH of the composition include acetate, citrate, prolamine, phosphate, carbonate, phthalate, borate, or other pharmaceutically acceptable buffers and mixtures thereof. In a particular example, the buffer comprises sodium phosphate. The pH of the composition is maintained generally to be compatible with the fluids of the nasal membrane in order to minimize irritation. The concentration of the buffer in the composition will depend upon the selection of the buffer and the desired pH.

The present composition may also contain various pharmaceutically acceptable additives such as tolerance enhancers (also known as humectants), absorption enhancers (also known as surfactants), preservatives, viscosity modifying agents (e.g., thickening agents), osmolarity adjusters, complexing agents, stabilizers, solubilizers, or any combination thereof.

A tolerance enhancer may be used in order to inhibit drying of the nasal membrane or mucosa. A tolerance enhancer may also serve the purpose of inhibiting or relieving irritation of the nasal membranes. Examples of suitable tolerance enhancers include, for example, humectants such as sorbitol, propylene glycol, glycerol, glycerin, hyaluronan, aloe, mineral oil, vegetable oil, soothing agents, membrane conditioners, sweeteners, and mixtures thereof. The selection and concentration of a tolerance enhancer may depend on a number of factors, including, for example, the concentration of ARM-1 compound being used in the composition.

A surfactant or absorption enhancer may also be used in the composition in order to enhance the absorption of the ARM-1 compound across the nasal membrane. Suitable absorption enhancers include non-ionic, anionic, and cationic surfactants. Any of a number of well-known surfactants may be used, including, for example, polyoxyethylene derivatives of fatty acids, partial esters of sorbitol anhydrides, sodium lauryl sulfate, sodium salicylate, oleic acid, lecithin, dehydrated alcohol, Tween (e.g., Tween 20, Tween 40, Tween 60, Tween 80 and the like), Span (e.g., Span 20, Span 40, Span 80 and the like), polyoxyl 40 stearate, polyoxy ethylene 50 stearate, edetate disodium, propylene glycol, glycerol monooleate, fusieates, bile salts, octoxynol and combinations thereof.

A pharmaceutically acceptable thickening agent may also be used in the composition in order to modify the viscosity of the composition. Numerous pharmaceutically acceptable thickening agents are well-known and include, for example, methyl cellulose, xanthan gum, carboxymethyl cellulose, hydroxypropyl cellulose, carbomer, polyvinyl alcohol, alginates, acacia, chitosans and combinations thereof. The concentration of the thickening agent will depend upon the agent selected and the viscosity desired.

A preservative may also be employed to increase the shelf-life of the composition. A number of well-known and pharmaceutically acceptable preservatives may be used in the present composition, including, for example, parabens, thimerosal, chlorobutanol, benzalkonium chloride, or benzyl alcohol and combinations thereof. Other ingredients which extend shelf life can be added such as for example, antioxidants. Examples of antioxidants include sodium metabisulfite, potassium metabisulfite, ascorbyl palmitate and other pharmaceutically acceptable antioxidants.

A suitable concentration of preservative will depend on a number of factors, including, for example, the particular preservative selected, the intended shelf-life of the composition, and the results of preservative effectiveness and minimum preservative studies.

Alternatively, the nasal spray may be formulated to be a sterile, preservative-free composition. While preservatives may extend the shelf life of a composition, they may also cause or exacerbate irritation to the nasal membranes.

### Buccal Tablet

Also provided herein, is a buccal tablet including ARM-1. A buccal tablet administered at the buccal cavity, the space between the cheek and the gum, is flanged on one side by the gum tissues and the other side by the cheek tissues or membranes, such as membranes in the mouth. The absorption of a drug, such as ARM-1 and its derivatives, in the buccal tissues and membrane begins the moment the drug comes out of the surface of the tablet or on the surface of the tablet itself. Such microscopic absorption of drug can be accelerated by a base (such as when the drug is a base) or an acid (such as when the drug is an acid). Similarly, the absorption of the drug can be decelerated by use of use a disintegrant and a buffer in combination with an acid (such as when the drug is a base) or a base (such as when the drug is an acid).

The buccal tablet can include an excipient (non-active ingredient) used as the carrier or filler or matrix material. Other adjuvants, such as disintegrants, glidants, diluents, or lubricants, or a combination thereof, can also be present, as well as the more conventional colorants, flavorings, sweeteners, or other organoleptically-effecting materials, or a combination thereof.

Pharmaceutical compositions disclosed in the application may be prepared, packaged, or sold in formulations suitable for oral administration. The formulations of the pharmaceutical compositions described herein may be prepared by any method known or hereafter developed. In general, preparation includes bringing the active ingredient into association with a carrier or one or more other additional components, and then, if necessary or desirable, shaping or packaging the product into a desired single-or multi-dose unit.

As used herein, “additional components” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; demulcents; buffers; salts; thickening agents; fillers; emulsifying agents; antioxidants; stabilizing agents; pharmaceutically acceptable polymeric or hydrophobic materials, as well as other components and agents.

A tablet comprising the drug may be made, for example, by compressing or molding the drug, optionally containing one or more additional components. Compressed tablets may be prepared by compressing, in a suitable device, the drug in a free-flowing form such as a powder or granular preparation, and then optionally mixing with one or more of a binder, a lubricant, a glidant, an excipient, a surface active agent and a dispersing agent. Molded tablets may be made by molding in a suitable device, a mixture of the drug, a pharmaceutically acceptable carrier, and at least sufficient liquid to moisten the mixtures.

Tablets may further comprise a sweetening agent, a flavoring agent, a coloring agent, a preservative, or some combination of these in order to provide pharmaceutically elegant and palatable preparations.

Hard capsules comprising the pharmaceutical agent may be made using a physiologically degradable composition, such as gelatin. Such hard capsules comprise the active ingredient, and may further comprise additional components including, for example, an inert solid diluent. Soft gelatin capsules comprising the pharmaceutical agent may also be made using a physiologically degradable composition, such as gelatin. Such soft capsules comprise the pharmaceutical agent, which may be mixed with water or an oil medium.

Tablets and pills of the present application can additionally be prepared with release-controlling coatings. Such a coating may be colored with a pharmaceutically accepted dye. The amount of dye and other excipients in the coating may vary. The coating generally comprises film-forming polymers such as hydroxy-propyl cellulose, hydroxypropylmethyl cellulose, cellulose ester, or ether, in acrylic polymer or a mixture of polymers. The coating solution is generally an aqueous solution that may further comprise propylene glycol, sorbitan monooleate, sorbic acid, or fillers such as titanium dioxide, a pharmaceutically acceptable dye.

The solid pharmaceutical compositions of the present application may further include diluents. Diluents for solid compositions include, for example, microcrystalline cellulose (e.g. AVICEL™), silicified microcrystalline cellulose, microfine cellulose, lactose, starch, pregelatinized starch, calcium sulfate, sugar, dextrates, dextrin, dextrose, dibasic calcium phosphate dihydrate, tribasic calcium phosphate, kaolin, magnesium oxide, maltodextrin, mannitol, dextrates (e. g. EMDEX™), hydrated dextrates, polymethacrylates (e.g. EUDRAGIT™), potassium chloride, powdered cellulose, sodium chloride, sorbitol and talc.

Solid pharmaceutical compositions of the present application may further include binders, e.g., acacia, alginic acid, carbomer (e.g., carbopol), carboxymethylcellulose sodium, dextrin, ethyl cellulose, gelatin, guar gum, hydrogenated vegetable oil, hydroxyethyl cellulose, hydroxypropyl cellulose (e.g., KLUCEL™), hydroxypropyl methyl cellulose (e.g. METHOCEL™), liquid glucose, magnesium aluminum silicate, maltodextrin, methylcellulose, polymethacrylates, povidone (e.g., KOLLIDON™, PLASDONE™), pregelatinized starch, sodium alginate and starch.

Solid pharmaceutical compositions of the present application may further include disintegrants such as alginic acid, carboxymethylcellulose calcium, carboxymethylcellulose sodium (e.g., AC-DI-SOL™, PRIMELLOSE™), colloidal silicon dioxide, croscarmellose sodium, crospovidone (e.g., KOLLIDON™, POLYPLASDONE™), guar gum, magnesium aluminum silicate, methyl cellulose, polacrilin potassium, powdered cellulose, pregelatinized starch, sodium alginate, sodium starch glycolate (e.g., EXPLOTAB™), hydroxypropylcellulose, methylcellulose, povidone or starch. Glidants, such as, colloidal silicon dioxide, magnesium trisilicate, powdered cellulose, starch, talc, and tribasic calcium phosphate may also be added.

Other pharmaceutical additives of the present application may include: (i) lubricants such as magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate; (ii) flavoring agents and flavor enhancers such as vanillin, ethyl vanillin, menthol, citric acid, fumaric acid ethyl maltitol, and tartaric acid; (iii) pharmaceutically acceptable colorants; (iv) artificial sweeteners such as polyhydric alcohols, e.g., sorbitol, mannitol, xylitol, saccharin, saccharin sodium, aspartame, sucralose and maltitol; and, (v) natural sweeteners, such as glucose, fructose, sucrose and the like.

In some examples, ARM-1 may be administered topically. As used herein, “topical administration” refers to administration onto any accessible body surface of any human or animal species, for example, the skin or mucosal epithelia. In certain examples of this application, “topical” refers to an external application to the skin epithelium. In some examples, the application can be directed to a composition for topical administration, wherein the composition includes one or more pharmaceutically acceptable excipients and a therapeutically effective amount of ARM-1.

The ARM-1 composition to be administered topically can take the form of a semi-solid preparation, such as a gel, paste, or ointment, a pourable preparation, such as a lotion, or a foam. s used herein, “semi-solid” is understood to refer to the rheological properties of the formulations themselves, such that the formulations will flow under an applied force but will remain in situ following application to any accessible body surface. As used herein, a “lotion” is a dermatological vehicle that is a pourable suspension of insoluble powder in a liquid. As used herein, a “gel” is a semi-solid vehicle that consists of a liquid phase that is constrained within a three-dimensional polymeric network. The polymeric network may be formed by chemical (covalent crosslinks) or physical (hydrogen bonds, Van der Waals forces) interactions between polymer chains (more correctly, between functional groups on polymer chains). Where the liquid phase is non-aqueous, the gel is an organogel. Oleogels are lipophilic gels whose bases typically consist of liquid paraffin with polyethylene or fatty oils gelled with colloidal silica or a long-chain fatty acid soap. As used herein, an “ointment” base is a semi-solid vehicle composed of hydrophobic constituents. Ointments can take the form of non-hydrocarbon ointment. Ointments related to the present application can be formulated to provide a non-greasy, cosmetically acceptable appearance. As used herein, a “paste” is an ointment with a high loading of insoluble solids (up to 50% by weight) that forms a structured particulate matrix. As used herein, a “foam” is a disperse system consisting of a three dimensional network of films in air. Foams have a high surface area and tend to spontaneous collapse unless stabilized.

“Pharmaceutically acceptable excipient” or “excipient” includes without limitation any inactive material that is combined with an ARM-1compound of the applicaiton in order to produce a drug dosage form for topical administration. The term “pharmaceutically acceptable excipient” is intended to include, but is not limited to, any solvents, penetration enhancing agents, antioxidants, stiffening agents (e.g., thickeners), ointment bases, protectives, adsorbents, demulcents, emollients, preservatives, moisturizers, buffers, adjuvants, bioavailability enhancers, carriers, glidants, sweetening agents, diluents, dye/colorants, flavor enhancers, solubilizers (including surfactants), wetting agents, dispersing agents, suspending agents, stabilizers and isotonic agents, which have been approved by a regulatory agency, such as for example, but is not limited to, the United States Food and Drug Administration, the European Medicines Agency or Health Canada, as being acceptable for use in a formulation for the topical administration of a pharmacologically active ingredient, and/or are considered as Generally Recognized As Safe materials (GRAS materials), and/or are listed in the Inactive Ingredients Guide published by the United States Food and Drug Administration. “Pharmaceutically acceptable excipient” can also comprise the acceptable excipients listed in Remington: The Science and Practice of Pharmacy. Exemplary pharmaceutically acceptable excipients include, but are not limited to, the following: ascorbic acid and esters; benzyl alcohol; benzyl benzoate; butylated hydroxytoluene (“BHT”); butylated hydroxyanisole (“BHA”); caprylic/capric triglyceride; cetyl alcohol; chelating agents (e.g., EDTA and citric acid); cholesterol; cross-linked acrylic acid based polymers (e.g., Carbopol™); decyl methyl sulfoxide; diethyl sebacate; dimethylamine (“DMA”); dimethicone; dimethyl sulfoxide; diethylene glycol mono ether (e.g., Transcutol™ P); diisopropyl adipate (e.g., Ceraphyl™ 230); ethanol; flavinoid; glutathione; glycerine; glycerol oleate/propylene glycol (e.g., Arlacel 186); glycerol monooleate; glyceryl caprylate/caprate and PEG-8 (polyethylene glycol) caprylate/caprate complex; carpylocaproyl macrogolglycerides (e.g., Labrasol™); glyceryl monocaprylate (e.g., Capmul™ MCM C8); glyceryl monolinoleate (e.g., Maisine™ 35-1); glyceryl monooleate (e.g., Peceol™); glyceryl monostearate; hexylene glycol; hydroxypropyl-β-cyclodextrin (HP-β-CD); isopropyl alcohol; isopropyl myristate; laurocapram; (e.g., Azone™); lauroyl macrogol-32 glycerides (e.g. Gelucire™ 44/14); macrogol-15 hydroxystearate (e.g., Solutol™ HS15); medium chain triglycerides (e.g., Miglyol™ 810, Miglyol™ 840 or Miglyol™ 812); methyl laurate; N-methyl-2-pyrrolidine (e.g., Pharmasolve™); mineral oil; mono diglycerides (e.g., Capmul™ MCM); octyldodecanol; oleic acid; oleyl alcohol; peanut oil; 1,2-pentanediol; polysorbates (e.g., Tween™ 80); polyethylene glycol (e.g., PEG-8, PEG 400, PEG1000, PEG 3350, PEG 6000, or Lutrol™ E 400); polyoxyl 35 castor oil (e.g., Cremophor™ EL); polyoxyl 40 hydrogenated castor oil (e.g., Cremophor™ RH 40); propylene glycol; propylene glycol diacetate; propylene glycol monocaprylate (e.g., Capmul PG-8, Capryol 90); propylene glycol monolaurate (e.g., Capmul PG-12); propylene glycol monooleate; 2-pyrrolidone; soybean oil; stearyl alcohol; sulfobutylether-β-cyclodextrin (e.g., Capitsol™); tocopherols (e.g., Vitamin E acetate); α-tocopherol polyethylene glycol succinate (TPGS); water; and white petrolatum.

“Solvents” refer to substances that readily dissolve other substances, such as ARM-1 in order to form a solution. Suitable solvents for the purposes of this application include polyethylene glycol (e.g., PEG 400, PEG 100, and PEG 3350), diethylene glycol monoethyl ether (e.g., Transcutol™), Tween 80, alcohols (e.g., oleyl alcohol, and stearyl alcohol), Labrasol™, caprylic/capric triglyceride, fatty acid esters (e.g., isopropyl myristate, and diisopropyl adipate (e.g., Ceraphyl™ 230)), diethyl sebacate, propylene glycol monocaprylate (e.g., Capmul™ PG-8), propylene glycol laurate (e.g., Capmul™ PG-12), mono diglycerides (e.g., Capmul™ MMCM), glyceryl monocaprylate (e.g., Capmul™ MCM C8), medium chain triglycerides, hexylene glycol, glyceryl mono-oleate (e.g., Peceol™), 1,2-pentanediol, octyldodecanol, glyceryl mono-linoleate (e.g., Maisine™ 35-1), isopropyl alcohol, glycerol oleate/propylene glycol (e.g., Arlacel™ 186), mineral oil, water, and glycerine.

“Penetration enhancing agents” refer to substances that increase the permeability of the skin or mucosa to a pharmacologically active ingredient, such as ARM-1, so as to increase the rate at which the active ingredient permeates through the skin or mucosa of a mammal. Suitable penetration enhancing agents for the purposes of this application include, but are not limited to, dimethyl sulfoxide (DMSO), decylmethylsulfoxide, laurocapram (e.g., AZONE™), pyrrolidones (e.g., 2-pyrrolidone, and N-methyl-2-pyrrolidine (PHARMASOLVE™)), surfactants, alcohols (e.g., oleyl alcohol), oleic acid, polyethylene glycol (e.g., PEG 400), diethylene glycol monoethyl ether (e.g., TRANSCUTOL™), and fatty acid esters (e.g., isopropyl myristate, methyl laurate, glycerol monooleate, and propylene glycol monooleate). A penetration enhancing agent may be used independently or more than one may be used in a pharmaceutical composition of the application.

“Ointment bases” refers to substances that function as a carrier and enhance penetration into the skin in order to deliver a pharmacologically active ingredient, such as ARM-1, to the area to be treated in the mammal. Suitable “ointment bases” for the purposes of this application include, but are not limited to, polyethylene glycols (e.g., PEG 400 and PEG 3350). An ointment base may be used independently or more than one may be used in a pharmaceutical composition of the application.

“Stiffening agents” refers to substances which increase the viscosity and/or physical stability of a pharmaceutical composition of the application. Suitable “stiffening agents” for the purposes of this application include, but are not limited to, stearyl alcohol, carbopols, dimethicone and polymers. A stiffening agent may be used independently or more than one may be used in a pharmaceutical composition of the application.

“Antioxidants” refers to substances which are capable of preventing the oxidation of another molecule. Suitable “antioxidants” for the purposes of this application include, but are not limited to, butylated hydroxytoluene (BHT), butylated hydroxyanisole (BHA), tocopherols (e.g., Vitamin E acetate), flavinoid, glutathione, ascorbic acid, and its esters, DMSO, and chelating agents (e.g., EDTA and citric acid).

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, and undecylenic acid.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, and polyamine resins.

In further examples, ARM-1 may be administered in gum. As used herein, gum refers to chewing gum containing a therapeutically effective amount of ARM-1, wherein the chewing gum can be used as a means of administering the ARM-1 to a subject. Gum as distributed to the subject can include a gum structure, which includes, but is not limited to, compositions ranging from and inclusive of compounded elastomer to finished gum, which may include compounded elastomer in addition to some compounding aids, master batch gum base, compounded elastomer in addition to some subsequent gum ingredients, compounded elastomer in addition to some gum base ingredients and some subsequent gum ingredients, gum base, gum base in addition to some subsequent gum ingredients, master batch finished gum, and finished gum.

Finished gum refers to a gum structure that is generally ready for preparation to distribute the product to the consumer. As such, a finished gum may still require temperature conditioning, forming, shaping, packaging, and coating. However, the gum composition itself is generally finished. Not all finished gums have the same ingredients or the same amounts of individual ingredients. By varying the ingredients and amounts of ingredients, textures, flavor, and sensations, among other things, can be varied to provide differing characteristics to meet the needs of users.

Gum can include a water soluble bulk portion, a water insoluble gum base portion, and one or more flavoring agents. The water soluble portion dissipates over a period of time during chewing. The gum base portion is retained in the mouth throughout the chewing process. A finished gum is typically ready for user consumption.

A “finished gum base”, as used herein, refers to a gum structure that includes a sufficient combination of gum base ingredients that need only be combined with subsequent gum ingredients to form a finished gum. A finished gum base is a chewable visco-elastic material that includes at least a viscous component, an elastic component, and a softener component. For example, a typical gum base may include elastomer, at least some of the filler, resin and/or plasticizer, polyvinyl acetate, and a softener (such as an oil, fat, or wax). Merely compounded elastomer without the addition of any softener, for example, would not be a finished gum base because it would not be considered useable in a finished gum structure because of its difficulty, if not impossibility, to chew.

Gum structures may include a vast number of ingredients in various categories. Systems and methods of the present application may be used to mix any and all known ingredients including, but not limited to, ingredients in the following ingredient categories: elastomers, bulking agents, elastomer plasticizers (which includes resins), elastomer solvents, plasticizers, fats, waxes, fillers, antioxidants, sweeteners (e.g., bulk sweeteners and high intensity sweeteners), syrups/fluids, flavors, sensates, potentiators, acids, emulsifiers, colors, and functional ingredients.

The insoluble gum base generally includes ingredients falling under the following categories: elastomers, elastomer plasticizers (resins or solvents), plasticizers, fats, oils, waxes, softeners, and fillers. The gum base may include from 5% to 95% by weight of a finished gum. In some examples, the gum base may include from 10% to 50%, or 20% to 30% by weight of the finished gum.

The water soluble portion of finished gum may include subsequent gum ingredients falling under the following categories: softeners, bulk sweeteners, high intensity sweeteners, flavoring agents, acids, additional fillers, functional ingredients, and combinations thereof. Softeners are added to the gum in order to optimize the chewability and mouth feel of the gum. High intensity sweeteners may also be present and are commonly used with sugarless sweeteners. Typically, high intensity sweeteners are at least 20 times sweeter than sucrose.

Natural and artificial flavoring agents may be used and combined in any sensorially acceptable fashion. Optional ingredients such as colors, functional ingredients and additional flavoring agents may also be included in gum structures.

### EXAMPLES

To further illustrate the principles of the present disclosure, the following examples are put forth so as to provide those of ordinary skill in the art with a complete disclosure and description of how the compositions and methods claimed herein are made and evaluated. They are intended to be purely for purposes of example and are not intended to limit the scope of the disclosure. These examples do not exclude equivalents and variations of the present invention which are apparent to one skilled in the art. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Compound Preparation

(5-formylfuran-2-yl)boronic acid (1.0 g, 7.15 mmol), 4-bromo-1-iodo-2-methylbenzene (1.63 g, 5.50 mmol), sodium carbonate (2 mL, 2 M solution), and bis(triphenylphosphine)palladium(II) dichloride (193 370 mg, 0.28 mmol) were added to pressure vessel and dissolved in dimethoxyethane (2 mL) and ethanol (3.5 mL). The solution was sparged with argon for 5 minutes, and the reaction was then sealed and heated to 65° C. for 12 hr. The reaction was then cooled to room temperature and diluted into ethyl acetate/H₂O (30 mL, 1:1). The organic layer was separated, and the aqueous layer was extracted with ethyl acetate (3×30 mL). The combined organic layers were dried over MgSO₄, filtered, and concentrated in vacuo. The crude product was purified by ISCO column chromatography eluting with 0 to 40% EtOAc in hexanes to afford the brown solid title compound 1 (1.13 g, 77% yield). ₁H NMR (400 MHz, CDCl3) δ 9.68 (s, 1H), 7.67 (d, J=8.4 Hz 1H), 7.46-7.41 (m, 2H), 7.33 (d, J=3.6 Hz 1H), 6.74 (d, J=3.6 Hz 1H), 2.53 (s, 3H).

To a solution of compound 1 (0.5 g, 1.89 mmol) in dichloromethane (8 mL) was added N,N-dimethylpropane-1,3-diamine (0.25 g, 2.45 mmol). After stirring 2 h at room temperature, sodium triacetoxyborohydride (0.44 g, 2.08 mmol) and acetic acid (0.8 mL) were added to reaction mixture. The reaction mixture was stirred for 16 hr at room temperature, quenched with sat. sodium bicarbonate (20 mL), extracted with dichloromethane (3×30 mL). The combined organic phase was dried over MgSO₄, filtered, and concentrated in vacuo. The crude product was purified by ISCO column chromatography eluting with 0 to 70% MeOH in dichloromethane to afford the yellow oil product (0.29 g, 44% yield). ₁H NMR (400 MHz, CD₃OD) δ 7.63 (d, J=8.4 Hz, 1H), 7.44 (s, 1H), 7.39 (dd, d, J=8.4, 1.6 Hz, 1H), 6.60 (d, J=3.6 Hz, 1H), 6.42 (d, J=3.6 Hz, 1H), 3.83 (s, 2H), 2.67 (t, J=7.2 Hz, 2H), 2.48 (s, 3H), 2.37 (t, J=7.2 Hz, 2H), 2.25 (S, 6H), 1.73 (p, J=7.6 Hz, 2H); LCMS calc'd for C17H23BrN2O [M+H]+: 351.3 measured 352.4.

### S. typhimurium Mfd Purification

S. typhimurium Mfd was purified by growing up HM3787 cells overnight from a single colony in LB+20 μg/mL chloramphenicol with agitation at 37° C. The next day 1 L of fresh LB+chloramphenicol was inoculated with 10 mL of overnight culture, incubated with agitation at 37° C. at optical density of 0.3 1 mM of IPTG was added and growth was continued for another 4 hours. Cells were then centrifuged at 5,000 rpm for 10 min and washed with PBS. Cell paste was then resuspended in 30 mL HisTrap lysis buffer (50 mM NaPO₄ pH=8.0; 0.5 mM DTT; 0.5 M NaCl; 5 mM imidazole pH=8.0) plus 2× Roche inhibitor proteases pills and homogenized. Cells were sonicated at midi-tip for 5 minutes at 50% amplitude with 30 second pulse intervals in an ice bath then centrifuged at 15,000 rpm for 30 minutes at 4° C. Supernatant was loaded on a His-Trap column (6 mL) in the same buffer, washed with NiWash Buffer (50 mM NaPO₄, 300 mM NaCl, and 40 mM imidazole pH 7.4), then eluted with NiElution Buffer (50 mM NaPO₄, 300 mM NaCl, and 150 mM imidazole pH 7.4). One 15 mL fraction was collected and dialyzed overnight in a Slide-A-Lizer (Thermo Scientific) against 1000 mL of TGED Buffer (10 mM Tris-HCl pH=8.0; 5% glycerol; 0.1 mM DTT; 0.1 mM EDTA; 50 mM NaCl). Dialysis material was centrifuged at 15,000 rpm for 30 minutes at 4° C. Using an AKTA system supernatant was loaded at 3× Heparin (1 mL) columns (GE) in TGED Buffer+50 mM NaCl buffer at 0.6 mL/min (pressure 0.5 mPa) and eluted by the same buffer with 1 M NaCl in linear gradient from 0% to 100% in 20 column volumes. Fractions were 1 mL during loading and 1 mL during separation. Peaks with a correct molecular weight from Heparin purification were diluted to 50 mL in TGED buffer without NaCl and loaded at MonoQ 5/50 column (GE) in TGED buffer+50 mM NaCl buffer at 2 mL/min (pressure 3.4 mPa) and eluted by the same buffer with 1 M NaCl in linear gradient from 0% to 100% in 20 column volumes. Fractions were 1 mL during loading and 0.5 mL during separation. Eluate was dialyzed overnight against 1000 mL of 10 mM RA Buffer (10 mM HEPES, 1 mM DTT, 50% glycerol, 1 mM EDTA, 500 mM KCl, and 40 mM MgCl₂ pH 8.0) in a Slide-A-Lyser. Resulting solution was aliquoted in 0.1 mL parts and flash-frozen in liquid nitrogen before being stored at −80° C.

### Translocase Assay

HM3418 (E. coli NM525 Δmfd+pRCB-Nluc+pUC19) and HM3419 (E. coli NM525 Δmfd+pRCB-Nluc+pUC19-ST19mfd) were grown overnight from single colonies in the presence of antibiotic selection. Overnight cultures were back-diluted the following morning to OD600 0.05, then grown in the presence or absence of ARM-1 at 37° C. until cultures reach OD600 0.50. In a 96-well plate, 80 μL of cells from each sample were combined with 100 μL LB and 20 μL of luminescence reporter substrate (NanoLuc, Promega). Luminescence of each well quantified on a BioTek SynergyNeo plate reader.

### Binding Affinity Analysis

Microscale thermophoresis was performed on a Monolith (NanoTemper). Purified Mfd was His-tagged labeled using NanoTemper's His-Tag Labeling Kit (MO-L018) according to manufacturer's protocol. Serial 1:2 dilutions were made of C5 starting with an end concentration of 1 mM in PBST buffer for a total of 16 dilutions. Labeled Mfd was then added to each dilution with an end concentration of 50 nM. MST experiments were performed under default settings apart from fluorescence intensity set to 100%. The raw data was imported into Prism 9 software and nonlinear regression was used to find the K_{d}.

### NADH-Coupled ATPase Assay

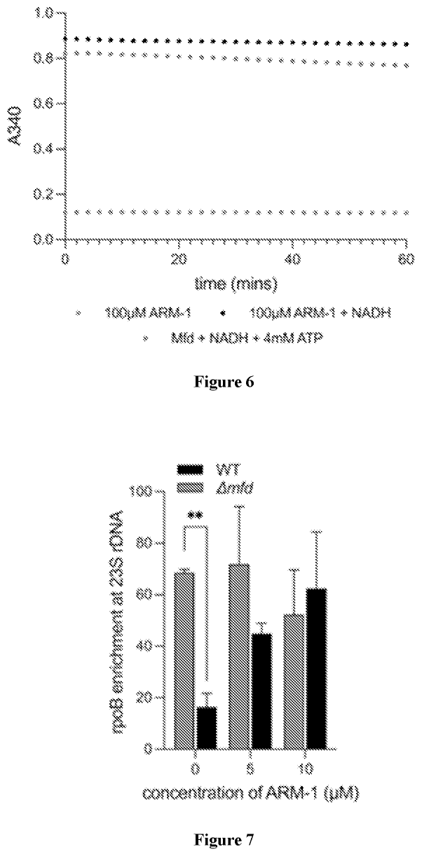

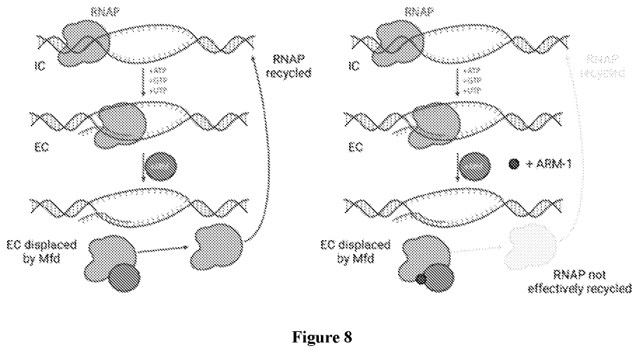

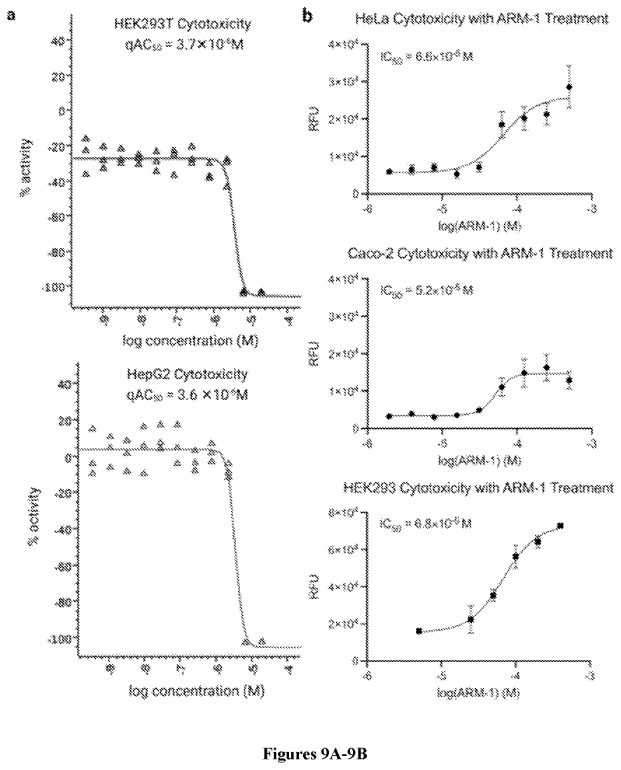

ATPase assays were performed as described in Kiiansita et al., 2003. All reactions were performed at 37° C. in a 150 μL reaction volume in a 96 well plate. Reactions were performed in repair buffer (40 mM HEPES pH 8.0, 100 mM KCl, 8 mM MgCl₂, 4% glycerol (v/v), 5 mM DTT and 100 μg/ml BSA) supplemented with 4.4 units pyruvate kinase, 5.7 units lactate dehydrogenase, 500 μM phosphoenolpyruvate and 400 μM NADH. Mfd in a final concentration of 50 nM and ARM-1 (aqueous solution, pH 8.0) in varying concentrations were added at least 15 minutes prior to starting reaction and allowed to incubate on ice. To start reaction, varying quantities of ATP were added and absorbance at 340 nm was measured every 60 seconds for 1 hour in an Epoch2 microplate spectrophotometer (BioTek). Results are in FIG. 6.

### RNAP Displacement Assay

5 ng of a ³²P labeled, 176 bp PCR fragment containing the promoter of the ampicillin resistance gene from pDR110, a gift from David Rudner, were incubated with 1 unit of E. coli RNA Polymerase holoenzyme (NEB) for 15 mins at 37° C. Then, NTPs were added to a final concentration of 1.7 mM (ATP) or 80 uM (UTP, GTP), as well as 80 uM ApU (Jena Bioscience), and incubated for 15 mins at 37° C. Purified S. typhimurium ST19 Mfd (final concentration of 100 nM) that had been pre-incubated for 10 mins at 37° C. with the indicated amounts of ARM-1 (aqueous solution, pH 8.0) was added and incubated for 6 mins at 37° C. 4 ul of the reaction were loaded into a polyacrylamide gel that had been pre-run for 45 mins at 70 V, and run for 55 mins at 150 V on ice, using 1× TBE buffer. The products were analyzed by phosphorimaging (GE Healthcare) and quantified using Image Lab 6.0.1.

### Evolution Assays

Evolution experiments were performed for the indicated strains. For S. typhimurium, S. aureus, L. monocytogenes, P. aeruginosa, and E. coli, overnight cultures, started from a single colony, were back diluted to OD600=0.005 and used to inoculate a 96-well plate. Cells were grown for 24 hours with agitation, at 37° C., in LB with a gradient of concentrations of the indicated antibiotic to select for resistance. Optical densities were subsequently measured in an Epoch2 microplate spectrophotometer (BioTek). Cultures that grew (defined by at least 50% growth relative to LB only) at the highest concentration of antibiotic were passaged into fresh LB with antibiotic in a subsequent plate. A total of 5 serial passages were performed. For P. aeruginosa, LB+0.01% tween 80 was used. For all species, antibiotics were diluted 2-fold down each given row in a 96 well plate. For sequencing of resistance loci from select evolution assays, gDNA was extracted from bacterial samples using GeneJet Genomic DNA Purification Kit (ThermoFisher). Samples were sequenced by Genewiz using custom primers.

### Mutagenesis Measurements Post Epithelial Cell Infection

HELA cells were cultured in DMEM supplemented with 20% heat-inactivated FBS, 1% glutamine, and 1% penicillin/streptomycin at 37° C. in 5% CO₂. The night before infection, 1×10⁷ HeLa cells were seeded into 15 cm plates and incubated overnight. A single S. typhimurium colony from strain HM1996 (WT St19) or HM3429 (DmfdSt19) was used to inoculate an overnight culture, grown in LB with appropriate antibiotic selection at 37° C. 260 rpm. The next morning, the overnight bacterial culture was set back to OD₆₀₀ 0.05 and grown to mid-exponential phase. The bacteria were then washed twice with tissue culture grade 1× PBS and resuspended in an appropriate volume of DMEM+20% FBS+1% glutamine. Bacteria were then applied to the HeLa cells and allowed to invade for 60 minutes at 37° C. 5% CO₂, at an MOI of 100:1. After 60 minutes of invasion, the bacteria were removed and fresh media applied to the HeLa cells. For ARM-1 treated conditions, the fresh media contained 31.25 μM ARM-1. 30 minutes later, 50 μg/mL gentamicin was added to the media to kill any remaining extracellular bacteria. After 8 hours of infection, HeLa cells were washed 2× with 1× PBS and lysed with 5 mL 1% Triton-X-100 in water. A small portion of lysate was serially diluted and plated on LB for CFU enumeration. The remaining volume was plated on LB+50 mg/mL 5-fluorocytosine (5-FC) and grown overnight at 37° C. to determine mutation frequency. Mutation frequency is reported as the ratio of number of colonies that grew on selection plates to the number of viable bacterial cells in each lysate.

### Mammalian Cell Cytotoxicity

Cytotoxicity of ARM-1 against HeLa, Caco-2, and HEK293 cells was determined using CellTox Green Cytotoxicity Assay (Promega) according to manufacturer instructions. ARM-1 was diluted in DMSO and applied to the culture such that final concentrations of DMSO were less than 0.05%. Reported toxicity is relative to a DMSO only control. Cells were incubated with ARM-1 for 24 hours prior to addition of reporter dye. Fluorescence was read on a BioTek Synergy Neo plate reader.

TABLE 1
depicts kinetic constants of Mfd ATPase activity.

| | |
|---|---|
| Vmax | km |

| | | | | |
|---|---|---|---|---|
| ARM-1 Treatment | mean | SEM | mean | SEM |

| | | | | | |
|---|---|---|---|---|---|
| 0 | μM | 2.771 | 0.343 | 0.954 | 0.319 |
| 62.5 | μM | 3.135 | 0.338 | 0.706 | 0.228 |
| 125 | μM | 4.170 | 0.711 | 1.470 | 0.583 |
| 250 | μM | 5.216 | 0.631 | 1.408 | 0.403 |
| 500 | μM | 7.401 | 0.666 | 1.576 | 0.322 |

Table 2 depicts resistance mutations arising in S. aureus
against trimethoprim, with and without ARM-1.

| WT | | | | | | WT + ARM-1 | | | | | |
|---|---|---|---|---|---|---|---|---|---|---|---|
| | | | | K185I | K185I | | | | | | |
| | | | K185I | K185I | K185I | | | | | | |
| | | | K185I | K185I | K185I | | | | | | |
| | | K1851 | M32T | M32T | M32T | | | | | | L358R |
| | | | K185I | K185I | K185I | | | | | | |
| 24 | 48 | 72 | 96 | 120 | 144 | 24 | 48 | 72 | 96 | 120 | 144 |

| | |
|---|---|
| Time (hours) | Time (hours) |

Table 3 depicts resistance mutations arising in S. typhimurium
against trimethoprim, with and without ARM-1.

| WT | | | | | | WT + ARM-1 | | | | | |
|---|---|---|---|---|---|---|---|---|---|---|---|
| | | | | | F153S | | | | | | |
| | | | | | D27E | | | | | | |
| | | | | D27E | D27E | | | | | | |
| | | P21L | P21L | P21L | P21L | | | | | | |
| | | F153S | F153S | F153S | F153S | | | | | | |
| | | F153S | D27E | D27E | D27E | | | | | | F153S |
| | | | F153S | F153S | F153S | | | | | P21L | P21L |
| 24 | 48 | 72 | 96 | 120 | 144 | 24 | 48 | 72 | 96 | 120 | 144 |

| | |
|---|---|
| Time (hours) | Time (hours) |

Table 4 depicts resistance mutations arising in S. aureus
against rifampicin, with and without ARM-1.

| WT | | | | | | WT + ARM-1 | | | | | |
|---|---|---|---|---|---|---|---|---|---|---|---|
| | | | D424G | D424G | D424G | | | | | | S439L |
| | | S482L | S482L | S482L | S482L | | | | R437C | R437C | R437C |
| | | | | D424G | D424G | | | | | | |
| | | D424N | D424N | D424N | D424N | | | | I480L | I480L | I480L |
| | | R437C | R437C | R437C | R437C | | | | | | |
| | | | | I480L | I480L | | | | | | |
| | I480M | I480M | I480M | I480M | I480M | | | | D424G | D424G | D424G |
| | | | Q421K | Q421K | Q421K | | | | | | L419S |
| | A430V | A430V | A430V | A430V | A430V | | | A430T | A430T | A430T | A430T |
| | | | Q421L | Q421L | Q421L | | | | | | S417P |
| | S417T | S417T | S417T | S417T | S417T | | A430V | A430V | A430V | A430V | A430V |
| | | D424R | D424R | D424R | D424R | | | | | | H434Y |
| 24 | 48 | 72 | 96 | 120 | 144 | 24 | 48 | 72 | 96 | 120 | 144 |

| | |
|---|---|
| Time (hours) | Time (hours) |

Table 5 depicts resistance mutations arising in L. monocytogenes
against rifampicin, with and without ARM-1.

| WT | | | | | | WT + ARM-1 | | | | | |
|---|---|---|---|---|---|---|---|---|---|---|---|
| | | | | Q470R | Q470R | | | | | | |
| | | | S488L | S488L | S488L | | | | | | |
| | | H483L | H483L | H483L | H483L | | | | | | G478D |
| | | Q470R | Q470R | Q470R | Q470R | | | | | H483L | H483L |
| 24 | 48 | 72 | 96 | 120 | 144 | 24 | 48 | 72 | 96 | 120 | 144 |

| | |
|---|---|
| Time (hours) | Time (hours) |

### REFERENCES

Dadgostar P. Antimicrobial Resistance: Implications and Costs. Infect Drug Resist. 2019;12:3903-3910. doi:10.2147/IDR.S234610.
Ragheb M N, Thomason M K, Hsu C, et al. Inhibiting the Evolution of Antibiotic Resistance. Mol Cell. 290 2019;73(1):157-165.e5. doi:10.1016/j.molcel.2018.10.015 291.
Westblade L F, Campbell E A, Pukhrambam C, et al. Structural basis for the bacterial transcription-repair 292 coupling factor/RNA polymerase interaction. Nucleic Acids Res. 2010;38(22):8357-8369. 293 doi:10.1093/nar/gkq692 294.
Park J S, Marr M T, Roberts J W. E. coli Transcription repair coupling factor (Mfd protein) rescues arrested 295 complexes by promoting forward translocation. Cell. 2002;109(6):757-767. doi:10.1016/s0092-296 8674(02)00769-9 297.
Chambers A L, Smith A J, Savery N J. A DNA translocation motif in the bacterial transcription-repair 298 coupling factor, Mfd. Nucleic Acids Res. 2003;31(22):6409-6418. doi:10.1093/nar/gkg868.
Du M, Kodner S, Bai L. Enhancement of LacI binding in vivo. Nucleic Acids Res. 2019;47(18):9609-9618. doi:10.1093/nar/gkz698.
Brugger C, Zhang C, Suhanovsky M M, et al. Molecular determinants for dsDNA translocation by the transcription-repair coupling and evolvability factor Mfd. Nat Commun. 2020;11(1):3740. doi:10.1038/s41467-020-17457-1.
Selby C P, Sancar A. Molecular mechanism of transcription-repair coupling. Science. 1993;260(5104):53-58. doi:10.1126/science.8465200.
Kiianitsa K, Solinger J A, Heyer W D. NADH-coupled microplate photometric assay for kinetic studies of ATP-hydrolyzing enzymes with low and high specific activities. Anal Biochem. 2003;321(2):266-271. doi:10.1016/s0003-2697(03)00461-5.
Kang J Y, Llewellyn E, Chen J, et al. Structural basis for transcription complex disruption by the Mfd 307 translocase. Elife. 2021;10:e62117. doi:10.7554/eLife.62117 308.
Smith A J, Pernstich C, Savery N J. Multipartite control of the DNA translocase, Mfd. Nucleic Acids Res. 309 2012;40(20):10408-10416. doi:10.1093/nar/gks775 310.
Song E, Uhm H, Munasingha P R, et al. Rho-dependent transcription termination proceeds via three routes. 311 Nat Commun. 2022;13(1):1663. doi:10.1038/s41467-022-29321-5 312.
Hao Z, Svetlov V, Nudler E. Rho-dependent transcription termination: a revisionist view. Transcription. 313 2021;12(4):171-181. doi:10.1080/21541264.2021.1991773 314.
Roberts J W. Mechanisms of Bacterial Transcription Termination. J Mol Biol. 2019;431(20):4030-4039. 315 doi:10.1016/j.jmb.2019.04.003.
Carter R H, Demidenko A A, Hattingh-Willis S, Rothman-Denes L B. Phage N4 RNA polymerase II recruitment to DNA by a single-stranded DNA-binding protein. Genes Dev. 2003;17(18):2334-2345. doi:10.1101/gad.1121403.
Smith A J, Pernstich C, Savery N J. Multipartite control of the DNA translocase, Mfd. Nucleic Acids Res. 2012;40(20):10408-10416. doi:10.1093/nar/gks775.
Ragheb M N, Merrikh C, Browning K, Merrikh H. Mfd regulates RNA polymerase association with hard-to-transcribe regions in vivo, especially those with structured RNAs. Proc Natl Acad Sci U S A. 2021;118(1). doi:10.1073/pnas.2008498118.
Richardson A R, Soliven K C, Castor M E, Barnes P D, Libby S J, Fang F C. The Base Excision Repair system of Salmonella enterica serovar typhimurium counteracts DNA damage by host nitric oxide. PLoS Pathog. 2009;5(5):e1000451. doi:10.1371/journal.ppat.1000451
Merrikh H, Kohli R M. Targeting evolution to inhibit antibiotic resistance. FEBS J. 2020;287(20):4341-4353. doi:10.1111/febs.15370.
Barlow M, Hall B G. Experimental prediction of the natural evolution of antibiotic resistance. Genetics. 336 2003;163(4):1237-1241.

### SEQUENCES

SEQ ID NO 1: Template Sequence:
TTAGACGTCAGGTGGCACTTTTCGGGGAAATGTGCGCGGAACCCCTAT
TTGTTTATTTTTCTAAATACATTCAAATATGTATCCGCTCATGAGACA
ATAACCCTGATAAATGCTTCAATAATATTGAAAAAGGAAGAGTATGAG
TATTCAACATTTCCGTGTCGCCCTTATTCCCT

1. A compound having a formula: 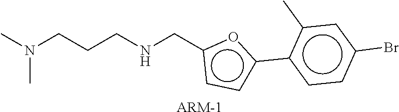
or a pharmaceutically acceptable salt or derivative thereof.

2. A method of reducing or preventing evolution of antimicrobial resistance in bacteria, comprising of administering to a subject a therapeutically effective amount of a compound having a formula: 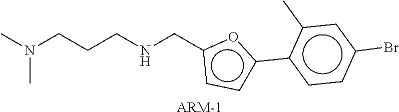
or a pharmaceutically acceptable salt or a derivative thereof.

3. The method of claim 2, wherein the bacteria is drug resistant.

4. The method of claim 2, wherein the bacteria is Staphylococcus aureus or Mycobacterium tuberculosis.

5. (canceled)

6. The method of claim 2, wherein the subject is a human.

7. (canceled)

8. The method of claim 2, wherein the compound inhibits evolvability factor RNA polymerase (RNAP)-associated DNA translocase.

9. The method of claim 8, wherein the evolvability factor RNA polymerase (RNAP)-associated DNA translocase is Mfd.

10. The method of claim 2, wherein the compound is administered in an in vitro setting.

11. The method of claim 2, wherein the compound is administered at a concentration of 50 to 100 μM.

12. The method of claim 2, wherein the compound is administered to the subject orally or buccally.

13. The method of claim 12, wherein the compound is in a tablet, troche, pill, or capsule.

14. (canceled)

15. The method of claim 2, wherein the compound is administered intravenously to the subject.

16. The method of claim 2, wherein the compound is administered to the subject as a spray.

17. The method of claim 16, wherein the compound is administered nasally.

18. (canceled)

19. The method of claim 2, wherein the compound is administered topically in an ointment, cream, lotion, solution, or tincture.

20. The method of claim 2, wherein the therapeutically effective amount of the compound is from 1 to 5,000 mg per day or from 1 to 1,000 mg/kg per day.

21. A pharmaceutical composition comprising the compound of claim 1 and an antibiotic drug.

22. The pharmaceutical composition of claim 21, wherein the antibiotic drug comprises penicillins, tetracyclines, cephalosporins, quinolones, lincomycins, macrolides, sulfonamides, glycopeptides, aminoglycosides, carbapenems, or any combination thereof.

23. The pharmaceutical composition of claim 21, wherein the pharmaceutical composition is a nasal spray.

24. The pharmaceutical composition of claim 21, wherein the pharmaceutical composition is a buccal tablet.


SEQUENCE LISTING (1 sequence)

SEQ ID NO: 1 - DNA, 176 bp, synthetic construct
  source 1..176: mol_type = other DNA
ttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattccct